(19) United States
(12) Patent Application Publication
(10) Pub. No.: US 2008/0160503 A1
(43) Pub. Date: Jul. 3, 2008

(54) RAPID ANALYSIS OF ORAL BIOAVAILABILITY

(76) Inventors: Levan DARJANIA, Escondido, CA (US); Sean O'Brien, Carlsbad, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/924,505

(22) Filed: Oct. 25, 2007

Related U.S. Application Data
(60) Provisional application No. 60/854,398, filed on Oct. 25, 2006.

(51) Int. Cl. C12Q 1/00; G01N 33/50; H01J 49/00

(52) U.S. Cl. 435/4; 436/173; 250/281

(57) ABSTRACT

Provided herein are procedures for rapidly determining oral bioavailability of two or more compounds by administering the two or more compounds to an animal, extracting fluid from an eye region of the animal and determining the amount of each of the two or more compounds in the fluid by mass spectrometry. Such procedures can provide cost advantages for determining oral bioavailability as they allow for (i) utilizing relatively small animals, (ii) extracting multiple samples from a single animal, and (iii) analyzing multiple compounds in a single animal, for example.

### RELATED APPLICATIONS

This application claims benefit of priority to U.S. Provisional Application Ser. No. 60/854,398, filed 25 Oct. 2006, the contents of which are incorporated herein by reference in their entirety.

### TECHNICAL FIELD

The invention is in general directed to processes and instrumentation for determining oral bioavailability of compounds.

### BACKGROUND ART

Discovering and developing new orally deliverable drugs is a complex and expensive process, which often ends in failure when a once promising drug candidate does not perform as expected in clinical trials, or is rejected by regulatory agencies. One reason these drug candidates fail is because of inappropriate bioavailability. When a drug is administered orally, before reaching the systemic circulation, it must move down the gastrointestinal tract and pass through the gut wall and liver, which are common sites of drug metabolism. This “first-pass metabolism” may destroy the drug before it can be measured in the systemic circulation. Drugs such as isoproterenol, norepinephrine, and testosterone have extensive first-pass metabolism, and their bioavailability through the oral route is virtually zero.

The term “bioavailability” refers to the extent to which, and sometimes the rate at which, the active moiety of a drug or metabolite enters systemic circulation, thereby gaining access to the site of action. Medications that are administered intravenously are considered to have 100 percent bioavailability, that is, the complete dose of the medication reaches the systemic circulation. But drugs that are administered through other routes, such as the oral route, generally do not have 100 percent bioavailability because these drugs may have various degrees of absorption.

Traditionally, bioavailability determination from plasma concentration-time data usually involves administering the compound to a human or other animal, withdrawing blood samples intravenously at certain times, and determining the maximum (peak) plasma drug concentration, the time at which maximum plasma drug concentration occurs (peak time), and the area under the plasma concentration-time curve (AUC). In oral dosing, the plasma drug concentration increases with the extent of absorption; the peak is reached when a “pseudo-equilibrium” exists between the drug elimination rate and the absorption rate. Because drug elimination begins once the drug enters the bloodstream, however, determining bioavailability solely based on peak plasma concentration may be misleading. Peak time is also used as an index for absorption rate, because slower absorption rates result in later peak times. Therefore, researchers often select AUC as a more reliable measure of bioavailability. For general information about bioavailability, see The Merck Manual of Diagnosis and Therapy (1995) 17th Ed. (Section 22, Chapter 298, “bioavailability” entry).

### SUMMARY

Provided hereafter are non-limiting examples of certain embodiments of the invention.

1. A method for determining oral bioavailability of a compound, which comprises:

(a) delivering two or more compounds or precursors thereof to an animal by oral administration;

(b) extracting fluid from an eye region of the animal; and

(c) determining the amount of each of the two or more compounds in the extracted fluid, or component thereof, by mass spectrometry, whereby the oral bioavailability of each compound is determined from the amount of each of the two or more compounds in the fluid.

2. The method of embodiment 1, wherein the fluid is blood.

3. The method of embodiment 2, wherein the blood is extracted from the retro-orbital region of the eye.

4. The method of embodiment 3, wherein the blood is extracted by retro-orbital puncture.

5. The method of any one of the preceding embodiments, wherein the fluid is extracted with a capillary tube.

6. The method of any one of the preceding embodiments, wherein the fluid is extracted at two or more time points.

7. The method of embodiment 6, wherein the time points are selected from the group consisting of about 15 minutes, about 30 minutes, about one hour, about two hours, about four hours, about six hours and about eight hours.

8. The method of any one of the preceding embodiments, wherein the animal is a rodent.

9. The method of embodiment 8, wherein the rodent is a mouse.

10. The method of embodiment 9, wherein the mouse is an ICR mouse.

11. The method of any one of the preceding embodiments, wherein the two or more compounds are small molecules.

12. The method of any one of the preceding embodiments, wherein the mass spectrometry is mass spectrometry/mass spectrometry (MS/MS).

13. The method of embodiment 12, wherein the MS/MS is coupled with liquid chromatography (LC-MS/MS).

14. The method of embodiment 13, wherein the liquid chromatography is high performance liquid chromatography.

15. The method of embodiment 12, wherein the MS/MS is coupled with electrospray ionization (ESI).

16. The method of embodiment 15, wherein the MS/MS is LC-ESI-MS/MS.

17. The method of any one of the preceding embodiments, wherein the amount of each of the two or more compounds is a concentration of each of the two or more compounds.

18. The method of any one of the preceding embodiments, wherein one or more parameters selected from the group consisting of Cmax, Tmax and AUC are determined from the amount of each of the two or more compounds.

19. The method of any one of the preceding embodiments, wherein three or more, four or more, five or more, six or more, seven or more, eight of more, nine or more, ten or more, fifteen or more, twenty or more, twenty-five or more, thirty or more, thirty-five or more, forty or more, forty-five or more or fifty or more compounds are administered to the animal.

20. The method of any one of the preceding embodiments, wherein the compounds are administered in a single formulation to the animal.

21. The method of any one of the preceding embodiments, wherein the extracted fluid is blood, and the component is a blood fraction.

22. The method of embodiment 21, wherein the blood fraction is blood plasma.

23. A mass spectrometer, which comprises a sample having two or more compounds, wherein the sample is derived from eye region fluid from an animal.

24. The mass spectrometer of embodiment 23, wherein the two or more compounds are small molecules.

25. The mass spectrometer of any one of the preceding embodiments, wherein the fluid is blood.

26. The mass spectrometer of embodiment 25, wherein the blood is from the retro-orbital region of the eye.

27. The mass spectrometer of embodiment 26, wherein the blood is extracted by retro-orbital venipuncture.

28. The mass spectrometer of any one of the preceding embodiments, wherein the animal is a rodent.

29. The mass spectrometer of embodiment 28, wherein the rodent is a mouse.

30. The mass spectrometer of any one of the preceding embodiments, which can be utilized to perform MS/MS analysis.

31. The mass spectrometer of any one of the preceding embodiments, which can be utilized to perform LC-MS/MS analysis.

32. The mass spectrometer of any one of the preceding embodiments, which can be utilized to perform LC-ESI-MS/MS analysis.

### DETAILED DESCRIPTION

Featured herein are procedures for rapidly determining oral bioavailability of a compound. These procedures can provide time and cost advantages for determining oral bioavailability of multiple compounds as they allow for (i) utilizing relatively small animals, (ii) extracting multiple samples from a single animal, and (iii) analyzing multiple compounds in a single animal, for example.

Thus, provided herein is a method for determining oral bioavailability of a compound, which comprises: (a) delivering two or more compounds or precursors thereof to an animal by oral administration; (b) extracting blood samples from an eye region of the animal; and (c) determining the amount of each of the two or more compounds in the extracted sample, or a component thereof, by mass spectrometry, whereby the oral bioavailability of each compound is determined from the amount of each of the compounds in the fluid. The term “oral bioavailability” as used herein refers to the extent to which, and sometimes the rate at which, a compound enters or is present in the blood sample from the eye region after oral administration of the compound or a precursor thereof to the animal. The term “determining oral bioavailability” as used herein refers to a process comprising assessing the amount of the compound in an eye region blood sample after the compound or precursor thereof is delivered by oral administration to the animal. The oral bioavailability is assessed for the animal administered the compound or precursor thereof, and oral bioavailability can then be modeled for other species (e.g., human subjects) based upon the information obtained. Oral bioavailability information for a compound can be ranked against like information obtained for other compounds, and can be utilized to determine pharmacokinetic parameters, described in greater detail hereafter. Oral bioavailability information obtained by methods described herein also can be compared to bioavailability information obtained by other methods (e.g., methods described in U.S. Provisional Patent Application No. 60/851,593, filed on Oct. 12, 2006).

Any animal can be utilized, such as a rodent (e.g., mouse, rat, guinea pig, rabbit), feline, canine, ungulate (e.g., bovine, equine, porcine, caprine), avian, reptile, amphibian, monkey or ape, for example. A mouse often is utilized, such as a genetically modified and/or immunodeficient mouse.

The two or more compounds can be small molecules. The term “small molecule” refers to an organic molecule having a molecular weight of about 1000 grams per mole or below. The term “compound” also can refer to a pro-drug, drug or metabolite of a compound administered to an animal. Any suitable number of compounds can be administered to the animal, such as three or more, four or more, five or more, six or more, seven or more, eight of more, nine or more, ten or more, fifteen or more, twenty or more, twenty-five or more, thirty or more, thirty-five or more, forty or more, forty-five or more or fifty or more compounds. The number of compounds and the particular compounds administered often are selected by the person of ordinary skill in the art based on the ability to detect the amount of each compound in a blood or fluid sample from the animal (an example of selection methods is described hereafter). The compounds can be administered separately to the animal, or can be administered as a single formulation (i.e., in a single dose) to the animal, the latter of which is referred to herein as a “cassette dose.”

A suitable oral formulation and oral delivery method can be readily selected by a person of ordinary skill in the art. In some embodiments, a compound or precursor is administered in combination with a pharmaceutically acceptable vehicle such as an inert diluent or an assimilable edible carrier. A compound or precursor may be enclosed in hard or soft shell gelatin capsules, compressed into tablets, or incorporated directly with the food of the animal's diet. For oral therapeutic administration, the active compound or precursor may be combined with one or more excipients and used in the form of ingestible tablets, buccal tablets, troches, capsules, elixirs, suspensions, syrups, wafers, and the like. Such compositions and preparations should contain at least 0.1% of active compound or precursor. The percentage of the compositions and preparations may be varied and may conveniently be between about 2 to about 60% of the weight of a given unit dosage form. Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound or precursor, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active compound or precursor may be incorporated into sustained-release preparations and devices.

Fluid may be extracted from a convenient region of the eye by a suitable method known to the person of ordinary skill in the art. The fluid often is blood and the blood sometimes is extracted from the retro-orbital region of the eye. The blood can be extracted by retro-orbital puncture, and a capillary tube may be utilized for the extraction. In certain embodiments, another fluid (e.g., urine) may be analyzed alone or in combination with fluid from the eye region. In certain embodiments, about 5 microliters to about 500 microliters of fluid is collected and analyzed by mass spectrometry. In particular embodiments, about 5 microliters to about 100 microliters of fluid, or about 5 microliters to about 50 microliters of fluid, or about 5 microliters to about 25 microliters of fluid, is extracted and analyzed by mass spectrometry. The extracted fluid may be processed prior to mass spectrometric analysis in some embodiments. For example, a blood fraction (e.g., blood plasma) may be separated from a blood sample extracted from an eye region, and the sample for mass spectrometric analysis may be prepared from the blood fraction (e.g., a mass spectrometric sample may be prepared from a blood plasma fraction).

The fluid can be extracted from an eye region at one or more time points. For example, fluid may be extracted at time points of about 15 minutes, about 30 minutes, about one hour, about two hours, about four hours, about six hours and/or about eight hours. Fluid may be extracted from one eye region or both eye regions. A blood sample also can be extracted from another anatomical site of an animal, such as a footpad, leg, back, hindquarter or neck of an animal.

Any suitable mass spectrometry instrument and method of analysis can be utilized to determine the amount of each compound in the fluid. Certain mass spectrometric analyses are available for detecting multiple compounds in a single sample. Such analyses can be performed in a concentration independent manner, and can be utilized to detect compounds present in each solution at a level of about 0.01 micromolar to about 10 micromolar (e.g., about one micromolar concentration). In certain embodiments, a mass spectrometer capable of performing mass spectrometry/mass spectrometry (MS/MS) analysis is utilized (i.e., capable of performing multiple reaction monitoring (MRM) analysis). Such mass spectrometers sometimes are utilized in conjunction with a liquid chromatography (LC) device, such as a high performance liquid chromatography (HPLC) device, for conducting LC-MS/MS analysis. In some embodiments, a MS/MS mass spectrometer is utilized in conjunction with an electrospray ionization (ESI) device, and sometimes such devices are utilized for LC-ESI-MS/MS analysis. Such mass spectrometers, LC and ESI devices are known to, and can be appropriately selected by, the person of ordinary skill in the art (e.g., Watanabe et al., Analytica Chimica Acta (2006) 559:37-44) for use in methods described herein.

The term “amount” as used herein refers to the quantity of one or more compounds in an eye region blood sample, or component thereof, from the animal. The amount can be determined for each compound in the sample, or can be determined for each compound in a subset of the compounds in the sample. The amount can be qualitative (e.g., area under a mass spectrometric peak) or quantitative (e.g., a concentration). A concentration of a compound may be expressed in any convenient manner, such as weight (e.g., milligrams or micrograms) or moles of a compound per unit volume (e.g., milliliters) or per unit weight of the fluid sample.

Based upon the amount of a compound in fluid samples taken at different time points, pharmacokinetic parameters also can be determined. The types of pharmacokinetic parameters and the methodology for determining the parameters can be appropriately selected by the person of ordinary skill in the art. Examples of pharmacokinetic parameters that can be assessed by methods described herein include, but are not limited to, maximum (peak) plasma drug concentration (Cmax), the time at which maximum plasma drug concentration occurs (peak time; Tmax), and the area under the fluid (e.g., plasma) concentration-time curve (AUC). The person of ordinary skill in the art is capable of calculating such parameters (e.g., Mei et al., AAPS Journal (2006) 8(3) article 58 (http address www.aapsj.org)).

Also provided herein is a mass spectrometer that comprises a sample having two or more compounds, wherein the sample is derived from eye region fluid from an animal. The two or more compounds are small molecules in certain embodiments, and the fluid can be blood. Blood from the retro-orbital region of the eye can be utilized, and the blood may be extracted by retro-orbital puncture. The animal often is a rodent and sometimes is a mouse. A mass spectrometer useful for determining the amount of two or more compounds in the sample often is selected by the person of ordinary skill in the art, such as a spectrometer that can be utilized to perform MS/MS analysis. In some embodiments, a mass spectrometer configuration for performing LC-MS/MS analysis is selected, and sometimes a mass spectrometer configuration for performing LC-ESI-MS/MS analysis is selected.

The Examples set forth hereafter illustrate certain embodiments of the invention and are not limiting. These Examples describe analyses of oral exposure for new chemical entities (NCEs) dosed in cassettes to ICR mice.

### EXAMPLE 1

### Cassette Design And Preparation

Compounds are selected for each cassette (i.e., cocktail) on the basis that spectrometric signals for each compound will not interfere with one another upon mass spectrometric analysis (e.g., will not overlap). The concentration of each compound in the dosing cassette is 20 mg/mL to achieve an oral dose level of 25 mg/kg in ICR mice.

MS/MS Method Development

Prepare 0.5 mL of 20 mg/mL dosing solution (in PBS or formulation vehicle) of 12 test compounds. Dilute a dosing solution 20 fold by transferring 10 μL of the stock solution into 190 μL acetonitrile containing 0.1% formic acid to achieve a final concentration of 1 mg/mL. Dilute further a 1 mg/mL solution 1,000 fold by transferring 1 μL of the stock solution into 999 μL acetonitrile containing 0.1% formic acid to achieve a final concentration of 1 μg/mL. Use the 1 μg/mL solution for mass spectrometric method development based on direct infusion. Determine parent/daughter mass spectra of each compound using multiple reaction monitoring (MRM). Compare parent/daughter mass spectra of each compound to assure no cross-reaction interference during LC-MS/MS measurements. Based on MRM mass spectra determine the composition of all cassettes.

Dosing Cassette Preparation

Mix 250 μL of four prepared dosing solutions and an oral PK internal standard (20 mg/mL each) according to cassette design to achieve a final concentration of 4 mg/mL. Vortex cassettes rigorously and ultra-sonicate to obtain a clear solution or homogeneous suspension. Use the cassette solutions to dose animals by oral route of administration at 25 mg/kg.

### EXAMPLE 2

### Administration of Cassettes to Animals

Animals and Dosing

All in vivo experiments follow protocols approved by the Animal Use and Care Committee. Female ICR mice (IcrTac:ICR), 8-10 weeks of age are obtained from Taconic (Hudson, N.Y.). Mice are housed on a 12 h/12 h light/dark cycle with ad libitum access to water and food. After a minimum two week acclimation period, the mice are randomized into groups with a minimum group size of four. The animals used for pharmacokinetic studies have a body-weight range of 25-35 g. A 25 mg/kg (4 mg/ml) dose of a cassette described in Example 1 is orally administered to mice that have been fasted overnight.

Blood Sample Preparation

After compound administration, serial blood samples are collected via retro-orbital puncture with a capillary tube at various time points (15, 30 minutes and 1, 2, 4, 6 and 8 hours). The samples are transferred to a heparinized 0.5 mL microcentrifuge tube and placed on ice. Plasma is separated by centrifugation and samples are stored at −80° C. until assay.

### EXAMPLE 3

### Example 3

### Bioanalysis

Preparation of Working Standard Solutions

Dilute a cassette dosing solution (4 mg/mL) four fold by transferring 25 μL of the stock solution into 75 μL of 50% acetonitrile containing 0.1% formic acid to achieve the concentration of 1 mg/mL. Dilute this stock solution further by serial dilutions to make 0.01, 0.1, 1 and 5 μg/mL working standard solutions.

Preparation of a Quenching Solution

Prepare 500 mL of 0.5 μg/mL solution of bioanalytical internal standard using 100% acetonitrile with 0.1% formic acid. Store the quenching solution in a tightly sealed bottle at 4C.

Calibration Standard Preparation for Analysis

Transfer 15 μL of blank mouse plasma to a 96 well plate and precipitate plasma proteins by pipetting of 120 μL of quenching solution to all plasma aliquots. Cover the plate with a matching plate mat and mix well for 30-60 seconds using a vertical multi-tube shaker.

Add 15 μL of the working standard solution of corresponding cassette to quenched plasma and vortex the plate for an additional 30-60 seconds. Then, centrifuge the plate at 4,000 rpm for 10 minutes at 4° C. Without disturbing the plasma protein pellet, transfer 120 μL of the supernatant to a new 96 well plate and dry the sample under nitrogen using a TurboVap® plate evaporator (Caliper Life Sciences; Hopkinton, Mass.). Reconstitute dried residues with 120 μL 20% acetonitrile containing 0.1% formic acid. Vortex the plate for 30-60 seconds and subject to LC-MS/MS analysis (described hereafter).

Study Sample Preparation for Analysis

Transfer 15 μL of blank mouse plasma to a 96 well plate and precipitate plasma proteins by pipetting of 120 μL of quenching solution to all plasma aliquots. Cover the plate with a matching plate mat and mix well for 30-60 seconds using a vertical multi-tube shaker.

Add 15 μL of the working standard solution of corresponding cassette to quenched plasma and vortex the plate for an additional 30-60 seconds. Add 15 μL of 50% acetonitrile containing 0.1% formic acid to quenched plasma to match the matrix and vortex the plate for an additional 30-60 seconds. Then, centrifuge the plate at 4,000 rpm for 10 minutes at 4° C. Without disturbing the plasma protein pellet, transfer 120 μl of the supernatant to a new 96 well plate and dry the sample under nitrogen using TurboVap® plate evaporator (Caliper Life Sciences; Hopkinton, Mass.). Reconstitute dried residues with 120 μL 20% acetonitrile containing 0.1% formic acid. Vortex the plate for 30-60 seconds and subject to LC-MS/MS analysis (described hereafter).

LC-MS/MS Analysis

Analyze the reaction mixtures for the amount of each chemical entity in the cassettes for the parent form according to the following HPLC conditions:

Column: Phenomenex Synergi Polar RP, 20.0×2.0 mm, 3 μM

Guard Column Phenomenex C18, 4.0×2.0 mm

Flow Rate: 0.25 mL/min

Column Temperature: 40° C.

Sample Temperature: 10° C.

Injection Volume: 10 μL

Run Time: 3.5 min

Gradient Solvent System:

Solvent A: 0.1% Formic Acid in Water

Solvent B: 0.1% Formic Acid in Acetonitrile

Solvent Gradient Profile:

| Time, | | |
|---|---|---|
| min | % A | % B |
| 0.0 | 95 | 5 |
| 1.0 | 95 | 5 |
| 1.5 | 5 | 95 |
| 2.0 | 5 | 95 |
| 2.5 | 95 | 5 |
| 3.5 | 95 | 5 |

Mass Spectrometry Parameters:

MS Mode: ESI (+)

Capillary: 3.5 kV

Cone: 40 V

Extractor: 3 V

RF Lens: 0.2 V

Source T: 120° C.

Desolvation T: 300° C.

Gas_Desolvation: 450 L/h

Gas_Cone: 72 L/h

LM Resolution: 15.0

HM Resolution: 15.0

Ion Energy: 0.5

Multiplier: 650

Pharmacokinetic Analysis

Apply noncompartmental pharmacokinetic analysis for oral administration. Record the observed Cmax and Tmax. Use linear trapezoidal rule to compute AUC (0-8) according to Gibaldi, M. and Perrier, D. Pharmacokinetics, Second Edition, Marcel Dekker, Inc., New York (1982).

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claim.

1. A method for determining oral bioavailability of a compound, which comprises:
(a) delivering two or more compounds or precursors thereof to an animal by oral administration;
(b) extracting fluid from an eye region of the animal; and
(c) determining the amount of each of the two or more compounds in the extracted fluid, or component thereof, by mass spectrometry, whereby the oral bioavailability of each compound is determined from the amount of each of the two or more compounds in the fluid.

2. The method of claim 1, wherein the fluid is blood.

3. The method of claim 2, wherein the blood is extracted from the retro-orbital region of the eye.

4. The method of claim 3, wherein the blood is extracted by retro-orbital puncture.

5. The method of claim 1, wherein a sample of the fluid is extracted at each of two or more time points and the oral bioavailability of each compound is determined from the amount of each of the two or more compounds in each sample at each time point.

6. The method of claim 5, wherein the time points are separated by an interval selected from the group consisting of about 15 minutes, about 30 minutes, about one hour, about two hours, about four hours, about six hours and about eight hours.

7. The method of claim 1, wherein the animal is a rodent.

8. The method of claim 7, wherein the rodent is a mouse.

9. The method of claim 8, wherein the mouse is an ICR mouse.

10. The method of claim 1, wherein the two or more compounds are small molecules.

11. The method of claim 1, wherein the mass spectrometry is mass spectrometry/mass spectrometry (MS/MS).

12. The method of claim 11, wherein the MS/MS is coupled with liquid chromatography (LC-MS/MS).

13. The method of claim 11, wherein the MS/MS is coupled with electrospray ionization (ESI).

14. The method of claim 1, wherein the amount of each of the two or more compounds is a concentration of each of the two or more compounds.

15. The method of claim 1, wherein one or more parameters selected from the group consisting of Cmax, Tmax and AUC are determined from the amount of each of the two or more compounds.

16. The method of claim 1, wherein three or more, four or more, five or more, six or more, seven or more, eight of more, nine or more, ten or more, fifteen or more, twenty or more, twenty-five or more, thirty or more, thirty-five or more, forty or more, forty-five or more or fifty or more compounds are concurrently administered to the animal.

17. The method of claim 1, wherein the compounds are administered in a single formulation to the animal.

18. The method of claim 1, wherein the extracted fluid is blood, and the component is a blood fraction.

19. The method of claim 18, wherein the blood fraction is blood plasma.

20. A mass spectrometer, which comprises a sample having two or more compounds, wherein the sample is derived from eye region fluid from an animal.

21. The mass spectrometer of claim 20, wherein the two or more compounds are small molecules.

22. The mass spectrometer of claim 20, wherein the fluid is blood.

23. The mass spectrometer of claim 22, wherein the blood is from the retro-orbital region of the eye.

24. The mass spectrometer of claim 20, which can be utilized to perform MS/MS analysis or LC-MS/MS analysis.

25. The mass spectrometer of claim 20, which can be utilized to perform MS/MS analysis or LC-MS/MS analysis.
